(19) United States
(12) Patent Application Publication
(10) Pub. No.: US 2026/0226200 A1
(43) Pub. Date: Aug. 6, 2026

(54) ANTIPOLYMERANT COMPOSITIONS AND METHODS FOR INHIBITING FOULING USING THE COMPOSITIONS

(71) Applicant: ECOLAB USA Inc., St. Paul, MN (US)

(72) Inventors: Bassam ALNASLEH, Sugar Land, TX (US); David DIXON, Baton Rouge, LA (US)

(73) Assignee: ECOLAB USA Inc., St. Paul, MN (US)

(21) Appl. No.: 19/457,892

(22) Filed: Jan. 23, 2026

Related U.S. Application Data
(60) Provisional application No. 63/754,248, filed on Feb. 5, 2025.

(51) Int. Cl. C08F 2/40; C07C 7/20; C08K 5/3435

(52) U.S. Cl. CPC C08F 2/40; C07C 7/20; C08K 5/3435

(57) ABSTRACT

The present disclosure provides compositions and methods for inhibiting or preventing fouling. A method of inhibiting polymerization of an ethylenically unsaturated monomer in a medium may include adding an effective amount of a composition to the medium. The composition includes a polymerization inhibitor, such as HTEMPO sebacate. Examples of ethylenically unsaturated monomers include acrylic acid, methacrylic acid, acrylonitrile, methacrylonitrile, acrolein, methacrolein, acrylate, methacrylate, acrylamide, methacrylamide, vinyl acetate, butadiene, ethylene, propylene, styrene, and any combination thereof.

### TECHNICAL FIELD

The present disclosure generally relates to compositions and methods for preventing or inhibiting fouling. More particularly, the disclosure relates to compositions and methods for inhibiting polymerization of reactive monomers.

### BACKGROUND

Fouling is an issue that may arise during production of various reactive monomers. An example is polystyrene fouling in quench and dilution steam units within ethylene production plants. Polystyrene fouling occurs throughout the quench and dilution steam system by the polymerization of styrene that is present in the process fluid. The dilution steam system is designed to recycle steam, which is added as a diluent to ethane and other feedstocks used in the production of ethylene. This steam is condensed along with pyrolysis gas and heavy components (pytar) in a quench tower. The quench tower flow is mostly water but contains percent levels of pyrolysis gas, which contains reactive monomers, such as styrene.

Styrene dissolved or emulsified in the aqueous stream can polymerize either by partitioning out of solution and polymerizing (bulk polymerization) or by emulsion polymerization or suspension polymerization. Traditional polymerization inhibitors used in dilution steam units have poor hydrocarbon solubility and typically partition with the aqueous phase and have little contact with styrene once it is precipitated or emulsified. Hydrophobic polymerization inhibitors have also been contemplated but not implemented because they have high volatility and have low thermal stability. Additionally, prior art inhibitors often include antioxidants, such as phenylenediamine, which require air or oxygen to work effectively.

### BRIEF SUMMARY

The present disclosure provides compositions and methods for preventing fouling/inhibiting polymerization of reactive monomers.

In some embodiments, the disclosure provides a method of inhibiting polymerization of an ethylenically unsaturated monomer in a medium. The method comprises adding an effective amount of a composition to the medium, the composition comprising a polymerization inhibitor of the following structure:

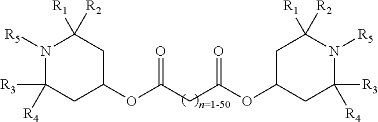

Each R₁, R₂, R₃, and R₄ is independently selected from a substituted or unsubstituted C₁-C₁₀ alkyl group, a C₁-C₁₀ alkenyl group, or a C₁-C₁₀ alkynyl group. Each R₅ is independently selected from H, OH, or O, and n is an integer from 1 to 50. The method also comprises the step of inhibiting polymerization of the monomer.

The foregoing has outlined rather broadly the features and technical advantages of the present disclosure in order that the detailed description that follows may be better understood. Additional features and advantages of the disclosure will be described hereinafter that form the subject of the claims of this application.

### DETAILED DESCRIPTION

Various embodiments are described below. The relationship and functioning of the various elements of the embodiments will be better understood in light of the following detailed description. However, elements and embodiments are not strictly limited to those explicitly described below.

Examples of methods and materials are described below, although methods and materials similar or equivalent to those described herein can be used in practice or testing of the present disclosure. All publications, patent applications, patents and other reference materials mentioned herein are incorporated by reference in their entirety. The materials, methods, and examples disclosed herein are illustrative only and not intended to be limiting.

Unless otherwise defined, all technical and scientific terms used herein have the same meaning as commonly understood by one of ordinary skill in the art. In case of conflict, the present document, including definitions, will control.

Unless otherwise indicated, an alkyl group as described herein alone or as part of another group is an optionally substituted linear or branched saturated monovalent hydrocarbon substituent containing from, for example, one to about sixty carbon atoms, such as one to about thirty carbon atoms, in the main chain. Examples of unsubstituted alkyl groups include methyl, ethyl, n-propyl, i-propyl, n-butyl, i-butyl, s-butyl, t-butyl, n-pentyl, i-pentyl, s-pentyl, t-pentyl, and the like.

The terms “aryl” or “ar” as used herein alone or as part of another group (e.g., arylene) denote optionally substituted homocyclic aromatic groups, such as monocyclic or bicyclic groups containing from about 6 to about 12 carbons in the ring portion, such as phenyl, biphenyl, naphthyl, substituted phenyl, substituted biphenyl or substituted naphthyl. The term “aryl” also includes heteroaryl functional groups. It is understood that the term “aryl” applies to cyclic substituents that are planar and comprise 4n+2 electrons, according to Huckel's Rule.

“Cycloalkyl” refers to a cyclic alkyl substituent containing from, for example, about 3 to about 8 carbon atoms, such as from about 4 to about 7 carbon atoms or about 4 to 6 carbon atoms. Examples of such substituents include cyclopropyl, cyclobutyl, cyclopentyl, cyclohexyl, cycloheptyl, cyclooctyl, and the like. The cyclic alkyl groups may be unsubstituted or further substituted with alkyl groups, such as methyl groups, ethyl groups, and the like.

“Heteroaryl” refers to a monocyclic or bicyclic 5- or 6-membered ring system, wherein the heteroaryl group is unsaturated and satisfies Huckel's rule. Non-limiting examples of heteroaryl groups include furanyl, thiophenyl, pyrrolyl, pyrazolyl, imidazolyl, 1,2,3-triazolyl, 1,2,4-triazolyl, isoxazolyl, oxazolyl, isothiazolyl, thiazolyl, 1,3,4-oxadiazol-2-yl, 1,2,4-oxadiazol-2-yl, 5-methyl-1,3,4-oxadiazole, 3-methyl-1,2,4-oxadiazole, pyridinyl, pyrimidinyl, pyrazinyl, triazinyl, benzofuranyl, benzothiophenyl, indolyl, quinolinyl, isoquinolinyl, benzimidazolyl, benzoxazolinyl, benzothiazolinyl, quinazolinyl, and the like.

Compounds of the present disclosure may be substituted with suitable substituents. The term “suitable substituent,” as used herein, is intended to mean a chemically acceptable functional group, preferably a moiety that does not negate the activity of the compounds. Such suitable substituents include, but are not limited to, halo groups, perfluoroalkyl groups, perfluoro-alkoxy groups, alkyl groups, alkenyl groups, alkynyl groups, hydroxy groups, oxo groups, mercapto groups, alkylthio groups, alkoxy groups, aryl or heteroaryl groups, aryloxy or heteroaryloxy groups, aralkyl or heteroaralkyl groups, aralkoxy or heteroaralkoxy groups, HO—(C═O)— groups, heterocylic groups, cycloalkyl groups, amino groups, alkyl- and dialkylamino groups, carbamoyl groups, alkylcarbonyl groups, alkoxycarbonyl groups, alkylaminocarbonyl groups, dialkylamino carbonyl groups, arylcarbonyl groups, aryloxy-carbonyl groups, alkylsulfonyl groups, and arylsulfonyl groups. In some embodiments, suitable substituents may include halogen, an unsubstituted C₁-C₁₂ alkyl group, an unsubstituted C₄-C₆ aryl group, or an unsubstituted C₁-C₁₀ alkoxy group. Those skilled in the art will appreciate that many substituents can be substituted by additional substituents.

The term “substituted” as in “substituted alkyl,” means that in the group in question (e.g., the alkyl group), at least one hydrogen atom bound to a carbon atom is replaced with one or more substituent groups, such as hydroxy (—OH), alkylthio, phosphino, amido (—CON(R_{A})(R_{B}), wherein R_{A} and R_{B} are independently hydrogen, alkyl, or aryl), amino (—N(R_{A})(R_{B}), wherein R_{A} and R_{B} are independently hydrogen, alkyl, or aryl), halo (fluoro, chloro, bromo, or iodo), silyl, nitro (—NO₂), an ether (—OR_{A} wherein R_{A} is alkyl or aryl), an ester (—OC(O)R_{A} wherein R_{A} is alkyl or aryl), keto (—C(O)R_{A} wherein R_{A} is alkyl or aryl), heterocyclo, and the like.

When the term “substituted” introduces a list of possible substituted groups, it is intended that the term apply to every member of that group. That is, the phrase “optionally substituted alkyl or aryl” is to be interpreted as “optionally substituted alkyl or optionally substituted aryl.”

The terms “polymer,” “copolymer,” “polymerize,” “copolymerize,” and the like include not only polymers comprising two monomer residues and polymerization of two different monomers together, but also include (co) polymers comprising more than two monomer residues and polymerizing together more than two or more other monomers. For example, a polymer as disclosed herein includes a terpolymer, a tetrapolymer, polymers comprising more than four different monomers, as well as polymers comprising, consisting of, or consisting essentially of two different monomer residues. Additionally, a “polymer” as disclosed herein may also include a homopolymer, which is a polymer comprising a single type of monomer unit.

Unless specified differently, the polymers of the present disclosure may be linear, branched, crosslinked, structured, synthetic, semi-synthetic, natural, organic, inorganic, and/or functionally modified. A polymer of the present disclosure can be in the form of a solution, a dry powder, a liquid, or a dispersion, for example.

The present disclosure provides compositions and methods for inhibiting fouling, such as fouling caused by polymerization of reactive monomers.

The technology disclosed herein leverages the unique physical and chemical properties of certain polymerization inhibitors, such as a 4-hydroxy-2,2,6,6-tetramethylpiperidin-1-yl)oxyl (HTEMPO) derivative, to address difficult to treat fouling that may arise during chemical manufacturing. The inventors determined that using a hydrophobic polymerization inhibitor, such as an HTEMPO derivative, alone or in combination with a traditional water-soluble polymerization inhibitor provides improved performance and fouling mitigation. The inventors also determined that the presently disclosed polymerization inhibitors have relatively high decomposition temperatures and relatively low vapor pressures as compared to other polymerization inhibitors used to inhibit polymerization of reactive monomers.

A low vapor pressure may include, for example, a vapor pressure less than about 1 torr at 100° C. For example, a low vapor pressure may be from about 0.001 to about 1 torr, such as about 0.01 to about 1 torr, about 0.1 to about 1 torr, or about 0.5 to about 1 torr.

A high decomposition temperature may be, for example, a temperature greater than about 175° C., greater than 200° C., greater than 225° C., greater than 250° C., greater than 300° C., greater than 400° C., etc. In some embodiments, a high decomposition temperature may be from about 175° C. to about 400° C., about 200° C. to about 400° C., or about 200° C. to about 300° C.

The compositions and methods disclosed herein effectively eliminate, inhibit, reduce, etc., reactive monomer (e.g., styrene) polymerization/fouling during various chemical manufacturing processes. For example, the compositions and methods are useful for inhibiting styrene polymerization and thus polystyrene fouling in the quench and dilution steam units within an ethylene production plant. The presently disclosed hydrophobic polymerization inhibitors, with low or no volatility, also have a high affinity for pygas, the main hydrocarbon component in a dilution steam system.

Fouling within a dilution steam system occurs due to the polymerization of styrene within the pygas component. The increased solubility of the presently disclosed hydrophobic inhibitors provides an advantage as they are present at higher concentrations within the pygas, which leads to less polymerization of styrene. Volatile polymerization inhibitors (e.g., diethyl hydroxyl amine), on the other hand, could return to the furnace where they will make NOx.

The inventors also discovered that the presently disclosed hydrophobic/hydrocarbon-soluble polymerization inhibitors offer improved performance where reactive monomers exist in a biphasic stream. Additionally, the use of a hydrophobic polymerization of the present disclosure in combination with one or more hydrophilic polymerization inhibitors could allow high concentrations of polymerization inhibitor to be present in both the hydrocarbon (pygas) and the aqueous streams within quench and dilution steam systems. Thus, while the present disclosure contemplates compounds and/or compositions comprising hydrophobic polymerization inhibitors, the disclosure also covers compounds and/or compositions comprising hydrophobic and hydrophilic polymerization inhibitors. Similarly, a hydrophilic polymerization may be added upstream and/or downstream of a point where a hydrophobic inhibitor is added.

A hydrophilic polymerization inhibitor may include, for example, DEHA, HTEMPO, 4-oxo-2,2,6,6-tetramethylpiperidinyl-1-oxyl (OTEMPO), or any combination thereof, for example

The inventors determined that using a hydrophobic polymerization inhibitor decreased polymerization of reactive monomers in mediums including oil, water, and one or more reactive monomers, which indicates that the presently disclosed polymerization inhibitors can effectively reduce fouling in quench towers, oil water separators, process water strippers, and other process equipment in a chemical manufacturing plant, such as an ethylene production plant.

A polymerization inhibitor of the present disclosure may comprise one or more compounds of the following structure:

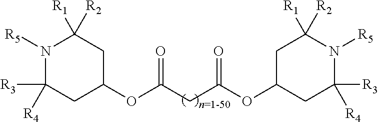

wherein each R₁, R₂, R₃, and R₄ is independently selected from a substituted or unsubstituted C₁-C₁₀ alkyl group, such as a C₁, a C₂, a C₃, a C₄, a C₅, a C₆, a C₇, a C₈, a C₉, or a C₁₀ alkyl group, a C₁-C₁₀ alkenyl group, such as a C₁, a C₂, a C₃, a C₄, a C₅, a C₆, a C₇, a C₈, a C₉, or a C₁₀ alkenyl group, or a C₁-C₁₀ alkynyl group, such as a C₁, a C₂, a C₃, a C₄, a C₅, a C₆, a C₇, a C₈, a C₉, or a C₁₀ alkynyl group; each R₅ is independently selected from H, OH, or O; and n is an integer from 1 to 50, such as 1 to 40, 1 to 30, 1 to 20, 1 to 10, 1 to 5, 5 to 50, 10 to 50, 20 to 50, 30 to 50, or 40 to 50.

In some embodiments, at least one of R₁, R₂, R₃ and R₄ is a methyl group. In some embodiments, at least two of R₁, R₂, R₃ and R₄ are methyl groups. In some embodiments, at least three of R₁, R₂, R₃ and R₄ are methyl groups. In some embodiments, each of R₁, R₂, R₃ and R₄ is a methyl group. In certain embodiments, each of R₁, R₂, R₃ and R₄ is the same substituent or a different substituent.

In illustrative embodiments, one or both of R₁ and R₂ may be selected from diethyl or cyclohexyl moieties, which are then reacted with sebacic acid to provide the corresponding sebacate. R₃, R₄, and R₅ are as defined above.

In some embodiments, a polymerization inhibitor of the present disclosure may have a pygas partition ratio of about 50,000:1 or greater, such as about 75,000:1, about 100,000:1, about 200,000:1, about 300,000:1, about 500,000:1, about 750,000:1, or about 1,000,000:1. The pygas partition ratio refers to the amount of a given compound present in pygas vs water after extraction. The partition ratio between the pygas and aqueous phase is calculated according to the formula P=(Concentration of inhibitor in the pygas)/(Concentration of the inhibitor in water). As an example, about 1-5 wt. % of inhibitor may be added to equal amounts of simulated pygas, shaken for about 30 seconds, and allowed to settle for about 30 minutes. The concentration may then be determined by analysis with ESR (electron spin resonance). The ratio allows for the differentiation of different inhibitors based on the partition coefficient. The higher the coefficient for a particular additive, the more will be present in the pygas or hydrocarbon phase.

A polymerization inhibitor may be added by itself (neat, without solvent) to a medium or it may be added in a composition with a solvent, such as a nonpolar solvent. Illustrative, non-limiting examples of solvents include dimethylphthalte, toluene, hexane, heavy aromatic distillate (HAD), heavy aromatic naphtha (HAN), or any combination thereof. A solvent may also include a polar aprotic solvent, acetonitrile, dimethylformamide, or any combination thereof.

A composition of the present disclosure may comprise any amount of solvent. For example, a composition may comprise from about 1 wt. % to about 99 wt. %, about 10 wt. % to about 99 wt. %, about 20 wt. % to about 99 wt. %, about 30 wt. % to about 99 wt. %, about 40 wt. % to about 99 wt. %, about 50 wt. % to about 99 wt. %, about 60 wt. % to about 99 wt. %, about 70 wt. % to about 99 wt. %, about 80 wt. % to about 99 wt. %, or about 90 wt. % to about 99 wt. % of the solvent.

Similarly, a composition of the present disclosure may comprise any amount of the polymerization inhibitor(s). For example, a composition may comprise from about 1 wt. % to about 99 wt. %, about 10 wt. % to about 99 wt. %, about 20 wt. % to about 99 wt. %, about 30 wt. % to about 99 wt. %, about 40 wt. % to about 99 wt. %, about 50 wt. % to about 99 wt. %, about 60 wt. % to about 99 wt. %, about 70 wt. % to about 99 wt. %, about 80 wt. % to about 99 wt. %, or about 90 wt. % to about 99 wt. % of the polymerization inhibitor(s). If a composition comprises a hydrophobic polymerization inhibitor and a hydrophilic polymerization inhibitor, each inhibitor may be present in any of the foregoing amounts.

The compositions of the present disclosure may include (or exclude) one or more additional components (other than the polymerization inhibitor) and/or one or more additional components may be added to the medium before, after, and/or with the polymerization inhibitor.

Illustrative, non-limiting examples of additional components include a corrosion inhibitor, a dispersant, an additional solvent, a passivator, or any combination thereof.

The corrosion inhibitor may comprise, for example, an imidazoline compound, a pyridinium compound, a quaternary ammonium compound, a phosphate ester, an amine, an amide, a carboxylic acid, a thiol, and any combination thereof.

The dispersant may comprise, for example, a methacrylate, a polyisobutylene succinic anhydride polymer, a tall oil fatty acid ester, a tetraethylenepentamine tall oil fatty acid, a soy oil fatty acid, a soy oil fatty acid ester, a coconut fatty acid ester, and any combination thereof.

In some embodiments, the dispersant is selected from a polyolefin polyamine, magnesium carbonate, an alkyl magnesium sulfonic acid magnesium salt, maleic anhydride with a C₂₄-C₂₈ alpha-olefin polymer, an alpha-olefin C₂₄-C₂₈ succinamide polymer), a polyolefin ester, an alkenyl succinimide in mineral oil, and any combination thereof.

The additional solvent may comprise, for example, a heavy aromatic naphtha, kerosene, a light aromatic (e.g., benzene, toluene, ethylbenzene, xylene) a glycol, an ether, a glycol ether, and any combination thereof.

The passivator may comprise, for example, phosphoric acid, mono(2-ethylhexyl) ester, a compound comprising C₁₂-C₁₄ tert-alkylamines, formaldehyde, a polymer comprising one or more of 4-(1,1-dimethylethyl)-1,2-benzenediol, dodecylphenol, and/or 1,2-ethanediamine, and any combination thereof.

The additional component may be added to the medium before, after, and/or with the polymerization inhibitor. In some embodiments, the additional component is added with the polymerization inhibitor. In some embodiments, the additional component is added separately (before and/or after) from the polymerization inhibitor.

The amount of additional component added to the medium is not particularly limited. For example, from about 1 ppm to about 5,000 ppm of the additional component may be added to the medium, such as about 1 ppm to about 2,500 ppm, about 1 ppm to about 2,000 ppm, about 1 ppm to about 1,500 ppm, about 1 ppm to about 1,000 ppm, about 1 ppm to about 750 ppm, about 1 ppm to about 500 ppm, about 1 ppm to about 250 ppm, about 1 ppm to about 100 ppm, about 25 ppm to about 5,000 ppm, about 25 ppm to about 2,500 ppm, about 25 ppm to about 1,500 ppm, about 25 ppm to about 1,000 ppm, or about 25 ppm to about 500 ppm.

If a composition of the present disclosure comprises the additional component, the composition may comprise from, for example, about 0.1 wt. % to about 25 wt. % of the component, such as from about 0.1 wt. % to about 20 wt. %, about 0.1 wt. % to about 15 wt. %, about 0.1 wt. % to about 10 wt. %, about 0.1 wt. % to about 5 wt. %, about 1 wt. % to about 5 wt. %, about 1 wt. % to about 10 wt. %, about 1 wt. % to about 15 wt. %, about 5 wt. % to about 15 wt. %, or about 5 wt. % to about 20 wt. %.

In accordance with the present disclosure, various methods are disclosed for inhibiting the polymerization of monomers, such as ethylenically unsaturated monomers. Ethylenically unsaturated monomers are well-known in the art and all ethylenically unsaturated monomers are intended to be covered by the present disclosure.

In some aspects, the ethylenically unsaturated monomer is selected from the group consisting of acrylic acid, methacrylic acid, acrylonitrile, methacrylonitrile, acrolein, methacrolein, acrylate, methacrylate, acrylamide, methacrylamide, vinyl acetate, butadiene, ethylene, propylene, styrene, and any combination thereof.

Methods of the present disclosure include adding an effective amount of a composition and/or polymerization inhibitor disclosed herein to a medium comprising a reactive monomer and inhibiting polymerization of the monomer. Polymerization may be inhibited by, for example, capturing carbon centered radicals formed thermally by antipolymerant chemistry (e.g., polymerization inhibitors). The composition comprises a polymerization inhibitor as described herein.

The medium comprising the reactive/ethylenically unsaturated monomer is not particularly limited and the methods and compositions disclosed herein can inhibit polymerization of the monomer regardless of the type of medium containing the monomer. As illustrative, non-limiting examples, a medium may comprise a fluid from the monomer manufacturing process, a fluid from a dilution steam unit, a fluid from a quench steam unit, a fluid from a cracking unit, and any combination thereof.

The amount of polymerization inhibitor added to the medium is not particularly limited and may be selected based on various factors, such as the amount of reactive monomer present in the medium. In some embodiments, the effective amount of polymerization inhibitor added to the medium is from about 1 ppm to about 10,000 ppm by weight of the ethylenically unsaturated monomer. Illustrative, non-limiting examples include about 1 ppm to about 5,000 ppm, about 1 ppm to about 2,000 ppm, about 1 ppm to about 1,000 ppm, about 1 ppm to about 800 ppm, about 1 ppm to about 600 ppm, about 1 ppm to about 400 ppm, about 1 ppm to about 250 ppm, about 1 ppm to about 200 ppm, about 1 ppm to about 100 ppm, about 1 ppm to about 75 ppm, about 1 ppm to about 50 ppm, about 1 ppm to about 25 ppm, about 10 ppm to about 100 ppm, or about 25 ppm to about 75 ppm of polymerization inhibitor added by weight of the ethylenically unsaturated monomer.

The compositions, compounds, and/or components disclosed herein may be added to the medium using a variety of different application methods known in the art. In some embodiments, the compositions, compounds, and/or components may added continuously or intermittently to the medium, either automatically or manually, by using, for example, chemical injection pumps. The chemical injection pumps may be programmed to add particular amounts of the compositions, compounds, and/or components at certain time intervals to the medium. In some aspects, the chemical injection pumps can be manually controlled to add particular amounts of the compositions, compounds, and/or components to the medium. In some embodiments, the addition may involve dripping, pouring, spraying, pumping, injecting, and/or otherwise adding the composition, compound, and/or component to the medium.

Any of the foregoing application methods may be independently selected to add a particular composition, compound, and/or component to the medium. Moreover, any of the forgoing application methods may be used to add a polymerization inhibitor and/or a component to the medium together or separately. For example, a chemical injection pump may be used to add a composition comprising a polymerization inhibitor and an additional component, such as a corrosion inhibitor, to the medium. As an additional example, the polymerization inhibitor may be added via a first chemical injection pump and an additional component may be added via a second chemical injection pump.

In some embodiments, a composition of the present disclosure may be added to a medium during a chemical manufacturing process, such as an ethylene manufacturing process. A composition of the present disclosure may be added to a medium where reactive monomers are present in a water circulation and/or recycling loop.

In some embodiments, the compositions and methods disclosed herein prevent or mitigate fouling (e.g., polymerized reactive monomers, polymer deposits, etc.) in a quench and/or dilution steam unit, in an oil water separator, in a process water stripper, or any other location or process equipment in a chemical manufacturing plant, such as an ethylene production plant.

The compositions of the present disclosure may be added before, after, and/or to any of the foregoing locations.

The foregoing may be better understood by reference to the following examples, which are intended for illustrative purposes and are not intended to limit the scope of the disclosure or its application in any way.

### EXAMPLES

15 ml of water and 3 mL of styrene were added to a 100 mL separatory funnel. To the separatory funnel was then added 100 μL of a 0.08 wt. % solution of each inhibitor or combination thereof. The separatory funnel was then shaken vigorously for 30 seconds and the water layer was discarded. 18 mL of fresh DI water was added and the process was repeated two more times. An additional 15 ml of DI water was added and the styrene and water were placed in a glass vial with a pressure relief cap. The vial was purged with nitrogen for 10 min using a syringe connected to a nitrogen line and perforated with a needle to allow nitrogen to escape. The vial was heated to 100° C. while being agitated with a vortex. The reaction was monitored to observe any sign of dispersed polystyrene. The onset of uninhibited polymerization was marked by the emergence of finely dispersed polystyrene particles which give a blue tinge to the color of the vial. The time from which the reaction temperature reached 100° C. to the appearance of suspended polystyrene was documented as the induction period.

During the investigation of the inhibition of polystyrene fouling in dilution steam systems, it was found that one mechanism of polystyrene buildup was through suspension polymerization, whereby polystyrene particles are suspended in water and form discrete polystyrene particles, which can accumulate and deposit in or on equipment which they form.

Numerous foulant analysis from various plants using prior art polymerization inhibitors still found polystyrene fouling in filters, process water strippers and dilution steam generators. Surprisingly, the addition of high levels of prior art inhibitors, such as HTEMPO, directly to any of these units had no affect on the fouling. This means that the foulant is likely generated upstream, such as in the quench tower, and carried forward. This fouling mechanism is best explained by suspension polymerization with a quench tower where the polystyrene is formed in the quench tower, suspended in the water, and the polystyrene particles are then carried forward and deposited.

The test carried out in accordance with these examples showed that HTEMPO sebacate significantly and unexpectedly outperform prior art inhibitors, such as HTEMPO, while having significantly less vapor, lower water solubility, and higher thermal stability than the prior art inhibitors. Specific data can be seen below in Table 1, wherein A=Blank, B=HTEMPO, C=HTEMPO sebacate, D=phenylenediamine, E=HTEMPO+HTEMPO sebacate, and E=HTEMPO+phenylenediamine.

TABLE 1

| | | | | | | | mmolal | mmolal | mmol | Total |
|---|---|---|---|---|---|---|---|---|---|---|
| Experiment | | Time 1 | Time 2 | Time 3 | Average | STDEV | HTEMPO | HTS | phenylenediamine | mmolal |
| Blank | A | 30 | 37 | 25 | 31 | 5 | 0.000 | 0.000 | 0.000 | 0.000 |
| HTEMPO | B | 70 | 65 | 75 | 70 | 4 | 0.026 | 0.000 | 0.000 | 0.026 |
| HTEMPO Sebacate | C | 130 | 120 | 138 | 129 | 7 | 0.000 | 0.008 | 0.000 | 0.008 |
| Phenylenediamine | D | 50 | 45 | 52 | 49 | 3 | 0.000 | 0.000 | 0.020 | 0.020 |
| HTEMPO + HTEMPO Sebacate | E | 90 | 100 | 87 | 92 | 6 | 0.013 | 0.004 | 0.000 | 0.017 |
| HTEMPO + Phenylenediamine | F | 90 | 93 | 85 | 81 | 3 | 0.013 | 0.000 | 0.010 | 0.023 |

Specifically, the present inventors determined that HTEMPO sebacate outperforms prior art inhibitors, such as HTEMPO, against suspension polymerization in a system where both water and styrene were present, and water was lost overtime or through blowdown such as in a quench and dilution steam system. HTEMPO, for example, is largely lost to the water once introduced and is not available in the monomer. In particular, HTEMPO would be carried downstream with the process water where it will continue to the process water stripper and dilution steam generator. Hydrophobic inhibitors like HTEMPO sebacate were found to particularly effective in suspension polymerization simulations within a dilution quench tower where monomer is polymerized, and the resulting polystyrene is suspended in the water phase. These suspended particles do not partition with the water layer but rather they are carried forward with the water and can foul downstream filters/coalescers, deposit in process water strippers, feed saturators, and/or dilution steam generators. In a system where water is the primary stream, such as a dilution steam system, it was found that HTEMPO sebacate was more effective at preventing the formation and suspension of polystyrene in simulations of a dilution steam system.

The vapor pressure and decomposition temperature seen below were determined with an isoteniscope using ASTM D2879.

| | | | | | | N,N′-di-sec- |
|---|---|---|---|---|---|---|
| Temper- | | | 4-acetoxyl | HTEMPO | | butylphenylene |
| ature | HTEMPO | OTEMPO | TEMPO | Sebacate | Diethyhydroxylamine | diamine |
| (° C.) | (torr) | (torr) | (torr) | (torr) | (DEHA) (torr) | (PDA) (torr) |
| 0 | 0.1 | 0.1 | 0.1 | 0.1 | 11 | 13 |
| 25 | 0.1 | 0.1 | 0.5 | 0.1 | 51 | 30 |
| 50 | 0.2 | 0.5 | 1.7 | 0.1 | 184 | 59 |
| 75 | 0.8 | 1.9 | 4.9 | 0.3 | 556 | 107 |
| 83 | | | | 0.9 | 760 | |
| 100 | 2.6 | 6.1 | 12 | | | 179 |
| 125 | 7.3 | 17 | 27 | 2.8 | | 280 |
| 150 | 18 | 43 | 54 | 7.2 | | 416 |
| 161 | | 62 | | | | |
| 175 | 41 | | 92 | 17 | | 592 |
| 193 | 70 | | | | | |
| 200 | | | | 36 | | 760 |
| 210 | | | | 47 | | |

It was determined that the decomposition temperature of HTEMPO was 193° C., OTEMPO was 161° C., 4-acetoxy TEMPO was 175° C., and HTEMPO sebacate was 210° C., while diethyhydroxylamine vaporized and N,N′-di-sec-butylphenylene diamine vaporized.

All of the compositions and methods disclosed and claimed herein can be made and executed without undue experimentation in light of the present disclosure. While this invention may be embodied in many different forms, there are described in detail herein specific preferred embodiments of the invention. The present disclosure is an exemplification of the principles of the invention and is not intended to limit the invention to the particular embodiments illustrated. In addition, unless expressly stated to the contrary, use of the term “a” is intended to include “at least one” or “one or more.” For example, “a polymerization inhibitor” is intended to include “at least one polymerization inhibitor” or “one or more polymerization inhibitors.”

Any ranges given either in absolute terms or in approximate terms are intended to encompass both, and any definitions used herein are intended to be clarifying and not limiting. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges (including all fractional and whole values) subsumed therein.

Any composition disclosed herein may comprise, consist of, or consist essentially of any element, component and/or ingredient disclosed herein or any combination of two or more of the elements, components or ingredients disclosed herein.

Any method disclosed herein may comprise, consist of, or consist essentially of any method step disclosed herein or any combination of two or more of the method steps disclosed herein.

The transitional phrase “comprising,” which is synonymous with “including,” “containing,” or “characterized by,” is inclusive or open-ended and does not exclude additional, un-recited elements, components, ingredients and/or method steps.

The transitional phrase “consisting of” excludes any element, component, ingredient, and/or method step not specified in the claim.

The transitional phrase “consisting essentially of” limits the scope of a claim to the specified elements, components, ingredients and/or steps, as well as those that do not materially affect the basic and novel characteristic(s) of the claimed invention.

Unless specified otherwise, all molecular weights referred to herein are weight average molecular weights and all viscosities were measured at 25° C. with neat (not diluted) polymers.

As used herein, the term “about” refers to the cited value being within the errors arising from the standard deviation found in their respective testing measurements, and if those errors cannot be determined, then “about” may refer to, for example, within 5%, 4%, 3%, 2%, or 1% of the cited value.

Furthermore, the invention encompasses any and all possible combinations of some or all of the various embodiments described herein. It should also be understood that various changes and modifications to the presently preferred embodiments described herein will be apparent to those skilled in the art. Such changes and modifications can be made without departing from the spirit and scope of the invention and without diminishing its intended advantages. It is therefore intended that such changes and modifications be covered by the appended claims.

What is claimed is:

1. A method of inhibiting polymerization of an ethylenically unsaturated monomer in a medium, comprising:
adding an effective amount of a composition to the medium, the composition comprising a polymerization inhibitor of the following structure: 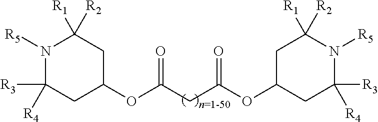
wherein each R₁, R₂, R₃, and R₄ is independently selected from a substituted or unsubstituted C₁-C₁₀ alkyl group, a C₁-C₁₀ alkenyl group, or a C₁-C₁₀ alkynyl group;
each R₅ is independently selected from H, OH, or O; and
n is an integer from 1 to 50; and
inhibiting polymerization of the monomer.

2. The method of claim 1, wherein at least one of R₁, R₂, R₃ and R₄ is a methyl group.

3. The method of claim 1, wherein each of R₁, R₂, R₃ and R₄ is a methyl group.

4. The method of claim 1, wherein the polymerization inhibitor comprises a pygas/partition ratio of about 50,000:1 or more.

5. The method of claim 1, wherein the composition comprises a polar solvent and/or a nonpolar solvent.

6. The method of claim 5, wherein the composition comprises from about 1 wt. % to about 99 wt. %.

7. The method of claim 1, wherein the composition comprises from about 1 wt. % to about 99 wt. % of the polymerization inhibitor.

8. The method of claim 1, further comprising adding a hydrophilic polymerization inhibitor to the medium.

9. The method of claim 1, further comprising adding an additional component to the medium before, after, and/or with the polymerization inhibitor.

10. The method of claim 9, wherein the additional component is selected from the group consisting of a corrosion inhibitor, a dispersant, an additional solvent, a passivator, and any combination thereof.

11. The method of claim 1, further comprising adding the composition during an ethylene manufacturing process.

12. The method of claim 1, further comprising adding from about 1 ppm to about 10,000 ppm of the polymerization inhibitor by weight of the ethylenically unsaturated monomer.

13. The method of claim 1, wherein the ethylenically unsaturated monomer is selected from the group consisting of acrylic acid, methacrylic acid, acrylonitrile, methacrylonitrile, acrolein, methacrolein, acrylate, methacrylate, acrylamide, methacrylamide, vinyl acetate, butadiene, ethylene, propylene, styrene, and any combination thereof.

14. The method of claim 1, wherein the composition is added continuously or intermittently to the medium.

15. The method of claim 1, wherein a decomposition temperature of the polymerization inhibitor is greater than about 175° C.

16. The method of claim 1, wherein the ethylenically unsaturated monomer comprises styrene and the polymerization inhibitor is added to a quench steam unit and/or a dilution steam unit within an ethylene production plant.
